(19) United States
(12) Patent Application Publication
(10) Pub. No.: US 2008/0082131 A1
(43) Pub. Date: Apr. 3, 2008

(54) Facial Nerve Stimulator (FNS)

(76) Inventor: Myriam Ivette Llanos, Carolina, PR (US)

Correspondence Address: MYRIAM IVETTE LLANOS, PO BOX 1468 VALLE ARRIBA HTS, CAROLINA, PR, 00984, US

(21) Appl. No.: 11/541,965

(22) Filed: Oct. 3, 2006

(51) Int. Cl. A61N 1/00

(52) U.S. Cl. 607/2

(57) ABSTRACT

The Facial Nerve Stimulator (FNS) is a headset type device used to treat patients with mild or severe Bell's Palsy and other facial nerve problems. The Facial Nerve Stimulator (FNS) is worn comfortably by resting one end on the ear lobe and inserting the Oral Electrode casing inside the mouth. The exterior Electrode casing rests just over the mandibular condyle. With the Oral Electrode casing inside the mouth and the exterior Electrode casing resting on the mandibular condyle, the FNS system is activated from a TENS unit to allow small electric pulses to stimulate both the 2nd branch (Maxillary) and the 3rd branch (mandibular) of the Trigeminal nerve as well as the mandibular condyle.

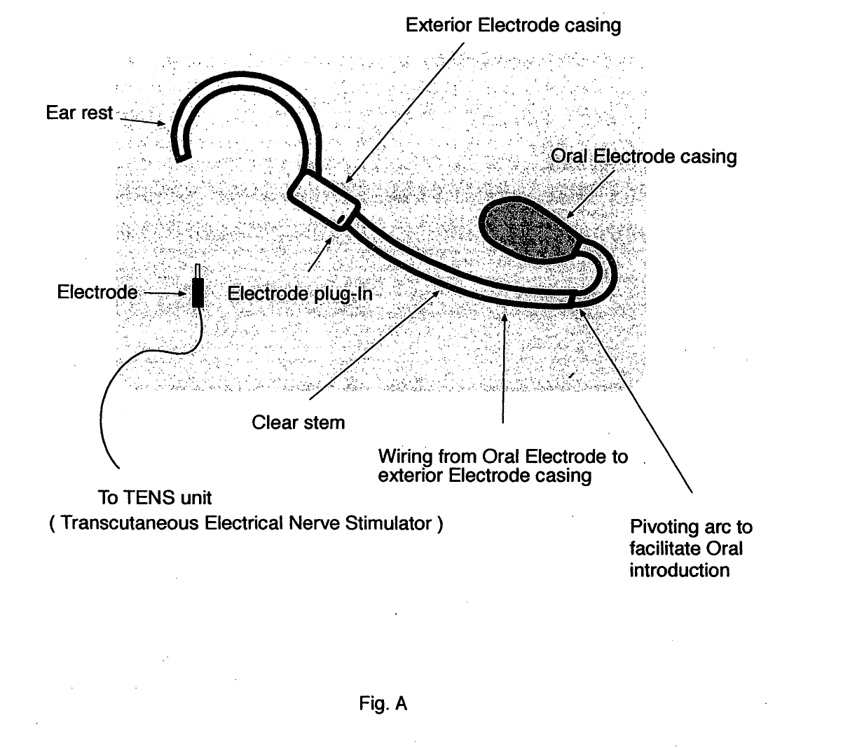

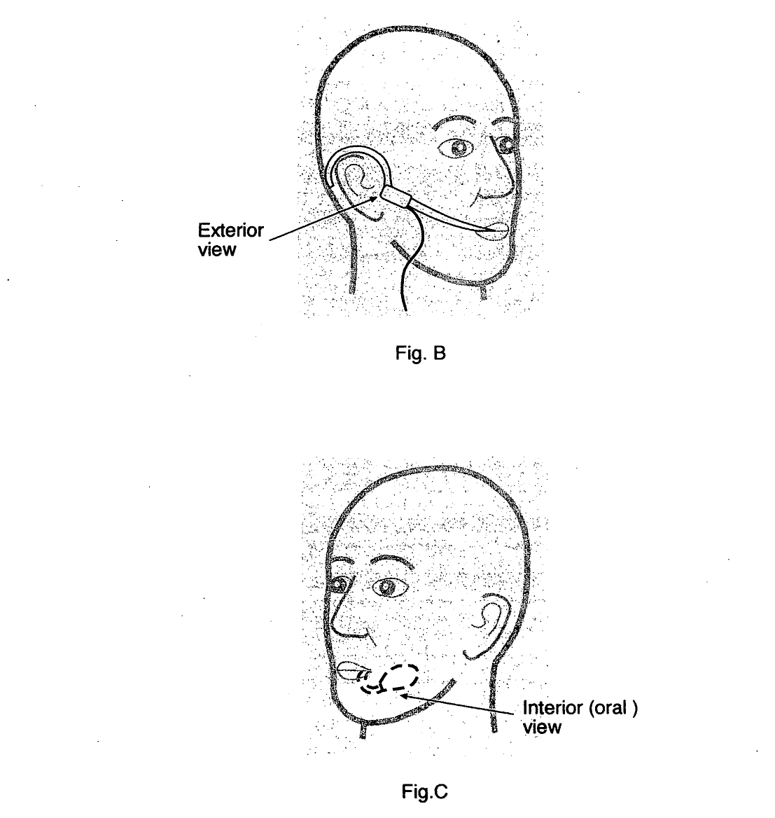

### BACKGROUND OF THE INVENTION

Having worked as a Dental Technician, the opportunity to invent the Facial Nerve Stimulator (FNS) arose when patients with mild or severe Bell's Palsy (facial paralysis caused by nerves) approached me with their particular problem. Given that most Neurologists prefer to treat this problem through certain drugs and my means of surgery, I felt another form of treatment was needed. This is when I invented the FNS.

### BRIEF SUMMARY OF THE INVENTION

The Facial Nerve Stimulator (FNS) is used for facial nerve stimulation therapy. This is for treating patients with mild or severe Bell's Palsy and other facial nerve problems including pain and discomfort. The Facial Nerve Stimulator (FNS) is worn comfortably by resting one end on the ear lobe and inserting the Oral Electrode casing inside the mouth. The exterior Electrode casing rests just over the mandibular condyle. With the Oral Electrode casing inside the mouth and the exterior Electrode casing resting on the mandibular condyle, the FNS system is activated from a TENS unit to allow small electric pulses to stimulate both the 2nd branch (Maxillary) and the 3rd branch (mandibular) of the Trigeminal nerve as well as the mandibular condyle.

### BRIEF DESCRIPTION OF THE SEVERAL VIEWS OF THE DRAWING

Figure A:

This is a side view of the Facial Nerve Stimulator (FNS). This includes the ear-rest that fits over the ear lobe area. This connects to the Exterior Electrode casing which houses the point of origin of the electric pulses generated by the TENS unit. The Interior Electrode casing, with its pivoting arc to facilitate oral introduction, will have wiring encased inside the stem all the way to the Exterior Electrode casing.

Figure B:

This view of the Facial Nerve Stimulator (FNS) shows the FNS being worn comfortably by resting one end on the ear lobe and inserting the Oral Electrode casing inside the mouth as shown. The exterior Electrode casing rests just over the mandibular condyle.

Figure C:

This view of the Facial Nerve Stimulator (FNS) shows the Oral Electrode casing inside the mouth. The FNS system is activated from a TENS unit to allow small electric pulses to stimulate both the 2nd branch (Maxillary) and the 3rd branch (mandibular) of the Trigeminal nerve as well as the mandibular condyle.

### DETAILED DESCRIPTION OF THE INVENTION

The Facial Nerve Stimulator (FNS) used for facial nerve stimulation therapy, will be manufactured using the latest material and state-of-the-art design. The FNS will be sleek in design and upholding the importance of an esthetic look so as to stand out as less as possible under the circumstances. A thin clear plastic stem will encase all wiring and the clear plastic will blend in with the patient's skin tone. The Facial Nerve Stimulator (FNS) is worn comfortably by resting one end on the ear lobe and inserting the Oral Electrode casing inside the mouth. The Oral Electrode casing will have a pivoting hook so as to bend the casing comfortably into the mouth. The exterior Electrode casing rests just over the mandibular condyle. With the Oral Electrode casing inside the mouth and the exterior Electrode casing resting on the mandibular condyle, the FNS system is activated from a TENS (Transcutaneous Electrical Nerve Stimulator) unit to allow small electric pulses to stimulate both the 2nd branch (Maxillary) and the 3rd branch (mandibular) of the Trigeminal nerve as well as the mandibular condyle. These pulses generayed by the TENS unit can be controlled to adjust the level of electric pulses needed for the particular therapy.

1. Myriam Lianos, claim as my invention an item called the Facial nerve Stimulator which will be used for facial nerve stimulation therapy. This is for treating patients with mild or severe Bell's Palsy and other facial nerve problems including pain and discomfort.
